(19) United States
(12) Patent Application Publication
(10) Pub. No.: US 2025/0069327 A1
(43) Pub. Date: Feb. 27, 2025

(54) IMPLANT LIBRARY MANAGEMENT METHOD, IMPLANT SIMULATION METHOD, AND DEVICE FOR IMPLEMENTING SAME

(71) Applicant: OSSTEM IMPLANT CO., LTD., Seoul (KR)

(72) Inventors: Sang Hyeong CHO, Gimpo-si (KR); Jong Moon KIM, Gunpo-si (KR); Kyoo Ok CHOI, Seoul (KR)

(21) Appl. No.: 18/726,115

(22) PCT Filed: Jan. 10, 2023
(86) PCT No.: PCT/KR2023/000417

§ 371 Date: Jul. 1, 2024

(30) Foreign Application Priority Data
Jan. 13, 2022 (KR) 10-2022-0005242

(51) Int. Cl. G06T 17/00; G16H 10/60

(52) U.S. Cl. CPC G06T 17/00; G16H 10/60

(57) ABSTRACT

A method performed by a computing device according to an embodiment of the present invention comprises the steps of: receiving specification information about an implant structure through a user input to a graphical user interface to which the specification information about the implant structure can be input; generating a 3D model of the implant structure by using the received specification information about the implant structure; and saving the generated 3D model of the implant structure in an implant library.

### TECHNICAL FIELD

The present disclosure relates to an implant library management method and a device for implementing the same.

### BACKGROUND ART

When planning and diagnosing implant procedures using medical imaging software or implant guide design software, a library of implant structures such as crowns, implants (the same term as “fixtures”), and abutments are loaded and placed into computerized tomography (CT) images or panoramic images taken inside the patient's oral cavity.

The library of implant structures stores specification information for known implant structures and, for example, 3D model data in stereolithography (STL) format. Building separate libraries for implants and abutments from each manufacturer is necessary, requiring the acquisition of specification information and 3D model data from each manufacturer.

In addition, when new implant products are released, the software's libraries need to be updated accordingly. However, if specification information and 3D model data for new products are not provided in a timely manner from the implant manufacturer, providing simulation functionality using the new products becomes difficult for software users.

Therefore, users who performs procedure planning and diagnosis for implants not provided in the software's library face limitations in conducting implant simulations using the specifications of actual implant structure.

### DETAILED DESCRIPTION OF INVENTION

### Technical Problem

The present disclosure is directed to providing an implant library management method for managing an implant library to enable the creation and registration of a three-dimensional (3D) model of an implant structure for which a 3D model is not provided in a library in software providing implant simulation functionality, and a device for implementing the same.

The present disclosure is also directed to providing an implant library management method that enables the provision of 3D models of implants and abutments by using specification information regarding the implants and abutments for which 3D models are not provided in software providing implant simulation functionality, and a device for implementing the same.

The present disclosure is also directed to providing an implant library management method capable of providing a convenient user interface to effectively manage an implant library for various types of implants and abutments in software providing implant simulation functionality, and a device for implementing the same.

Objectives of the present disclosure are not limited to the above-mentioned objectives, and other unmentioned objectives should be clearly understood by those of ordinary skill in the art to which the present disclosure pertains from the description below.

### Technical Solution

One embodiment of the present disclosure provides a method performed by a computing device, the method including the steps of: receiving specification information regarding an implant structure through a user input on a graphical user interface (GUI) to which the specification information regarding the implant structure can be input; generating a three-dimensional (3D) model of the implant structure by using the received specification information regarding the implant structure; and saving the generated 3D model of the implant structure in an implant library.

In one embodiment, the implant structure may include an implant, and the receiving of the specification information regarding the implant structure may include receiving the user input for entering a coronal diameter and a length of the implant.

In one embodiment, the receiving of the specification information regarding the implant structure may further include receiving a user input for entering the apical diameter of the implant to create the implant in a tapered shape.

In one embodiment, the receiving of the specification information regarding the implant structure may further include receiving a user input for entering a height of a hexagonal-shaped hex protruding at a top when the implant is of an external joint or butt joint type.

In one embodiment, the implant structure may include an abutment, and the receiving of the specification information regarding the implant structure may include receiving a user input for entering a reference diameter of the abutment, a length from the height of the reference diameter to the most coronal end, a length from the height of the reference diameter to the most apical end, and an axial inclination angle.

In one embodiment, the receiving of the specification information regarding the implant structure may include receiving a user input for entering a gingival angle for coupling to a lower part of the abutment, rather than receiving an input for a length from the height of the reference diameter to the most apical end.

In one embodiment, the receiving of the user input for entering the gingival angle for coupling to the lower part of the abutment may include receiving an input for an angle of a cylindrical cone connecting the outermost part of the uppermost circle of the implant and the outermost part of the diameter of the abutment.

In one embodiment, the method may further include displaying notification information when a position of a diameter area of the abutment changes according to the input gingival angle.

In one embodiment, the method may further include changing a state of a diameter area of the abutment to any one of an equigingival margin located at the same level as the top of gingiva, a supragingival margin located above the top of the gingiva, and a subgingival margin located below the top of the gingiva, according to the input gingival angle.

In one embodiment, the receiving of the specification information regarding the implant structure may further include receiving a user input for entering an abutment angle and an angle of inclination of an abutment to create the abutment in a shape that is asymmetrical in the left and right sides.

In one embodiment, the saving of the generated 3D model of the implant structure in the implant library may include registering the specification information regarding the implant structure and the 3D model in a pre-stored implant library, and displaying the registered specification information regarding the implant structure and the registered 3D model on a list of the implant library.

Another embodiment of the present disclosure provides an implant simulation device including at least one processor, a communication interface configured to communicate with an external device, a memory configured to load a computer program executed by the processor, and a storage configured to store the computer program, wherein the computer program includes instructions to perform an operation of receiving specification information regarding an implant structure through a user input on a graphic user interface (GUI) to which the specification information regarding the implant structure can be input, an operation of generating a three-dimensional (3D) model of the implant structure by using the received specification information regarding the implant structure, and an operation of saving the generated 3D model of the implant structure in an implant library.

### Advantageous Effects

As described above, according to the present disclosure, it is possible to create and register a 3D model of using specification information for an implant structure for which a 3D model is not provided in a library, thereby enabling utilization in implant simulation in software providing implant simulation functionality.

Additionally, according to the present disclosure, in software providing implant simulation functionality, a convenient user interface can be provided to effectively manage an implant library for various types of implants and abutments.

### DESCRIPTION OF DRAWINGS

FIG. 1 is a diagram illustrating the configuration of a system according to an embodiment of the present disclosure.

FIG. 2 is a block diagram illustrating the configuration of an implant simulation device according to an embodiment of the present disclosure.

FIGS. 3 and 4 are flowcharts illustrating an implant simulation method according to another embodiment of the present disclosure.

FIG. 5 illustrates an example of an implant library display screen according to some embodiments of the present disclosure.

FIG. 6 illustrates an example of a screen for entering information to create a custom implant of an internal joint type according to some embodiments of the present disclosure.

FIG. 7 illustrates an example of a screen for entering information to register a custom implant in a tapered shape according to some embodiments of the present disclosure.

FIG. 8 illustrates an example of a screen for entering information to create a custom implant of an external joint type according to some embodiments of the present disclosure.

FIG. 9 illustrates an example of a screen for entering information for creating a custom abutment of an internal joint type according to some embodiments of the present disclosure.

FIG. 10 illustrates an example where the gingival angle changes when the length from the diameter of an abutment to the most coronal end of an implant changes according to the gingival angle, or when the length from a set diameter of the abutment to the most coronal end of the implant changes.

FIG. 11 illustrates an example showing the types of abutment margins according to some embodiments of the present invention.

FIG. 12 illustrates an example of a screen for entering information to create a custom abutment of an external joint type according to some embodiments of the present disclosure.

FIG. 13 illustrates an example of a screen for entering information to create a custom abutment in a shape that is asymmetrical in the left and right sides according to some embodiments of the present disclosure.

FIG. 14 illustrates an example of a screen for registering a 3D model of a virtual implant in a library according to some embodiments of the present disclosure.

FIG. 15 illustrates an example of an interface screen for generating a 3D model of an implant structure according to some embodiments of the present disclosure.

FIG. 16 is a diagram illustrating a hardware configuration of an exemplary computing device by which methods according to some embodiments of the present disclosure can be implemented.

### MODE OF THE INVENTION

Hereinafter, example embodiments of the present disclosure will be described in detail with reference to the accompanying drawings. The advantages and features of the present disclosure and the manner of achieving the advantages and features will become apparent with reference to embodiments described in detail below together with the accompanying drawings. However, the present disclosure may be implemented in many different forms and should not be construed as being limited to the embodiments set forth herein, and the embodiments are provided such that this disclosure will be thorough and complete and will fully convey the scope of the present disclosure to those skilled in the art, and the present disclosure is defined only by the scope of the appended claims.

It is noted that the same or similar components in the drawings are designated by the same reference numerals as far as possible even if they are shown in different drawings. Also, in the following description of the present disclosure, a detailed description of known functions and configurations incorporated herein will be omitted to avoid making the subject matter of the present disclosure unclear.

Unless otherwise defined, all terms including technical or scientific terms used in this specification have the same meaning as commonly understood by those of ordinary skill in the art to which the present disclosure pertains. Also, terms defined in commonly used dictionaries should not be construed in an idealized or overly formal sense unless expressly so defined herein. Terms used in this specification are for describing the embodiments and are not intended to limit the present disclosure. In this specification, a singular expression includes a plural expression unless the context clearly indicates otherwise.

It will be understood that the terms “comprises” and/or “comprising,” when used herein, specify the presence of stated components, steps, operations and/or elements, but do not preclude the presence or addition of one or more other components, steps, operations and/or elements.

Hereinafter, some embodiments of the present disclosure will be described in detail with reference to the accompanying drawings.

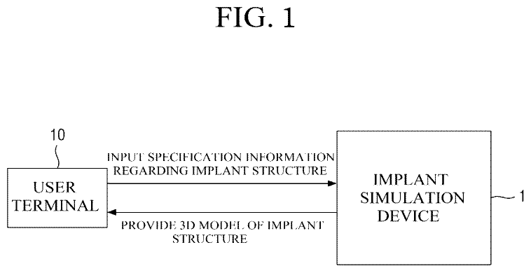

FIG. 1 is a diagram illustrating the configuration of a system according to an embodiment of the present disclosure. Referring to FIG. 1, an implant simulation device 1 according to an embodiment of the present disclosure provides an interface for receiving specification information regarding a custom implant structure through user input from a user terminal 10, generating a three-dimensional (3D) model of the custom implant structure, and updating an implant library by registering specification information and 3D model of a generated virtual implant in an implant library. The specification information regarding the custom implant structure may include parameters related to shape elements of the implant.

The implant simulation device 1 provides an interface screen for planning and simulating implant procedures using the computerized tomography (CT) image and panoramic image of a patient stored in the device or received from an external device (not shown).

When performing implant simulation, the implant simulation device 1 may load a library of an implant structure to be applied to the patient and place an individual 3D model registered in the library onto the CT image or panoramic image.

When a 3D model for an implant structure is not stored in the library, the implant simulation device 1 may provide a graphical user interface (GUI) screen to receive specification information for the custom implant structure, generate a 3D model from it, and register the custom implant structure in the implant library.

The implant simulation device 1 receives information regarding the specifications of a custom implant structure through user input on the GUI from a user terminal 10. The custom implant structure may be, for example, an implant or an abutment.

The implant simulation device 1 generates a 3D model of the custom implant structure utilizing the received specification information regarding the custom implant structure, and saves both the generated 3D model and specification information for the custom implant structure in the implant library.

With the system configuration of the present disclosure as described above, a 3D model of a custom implant structure may be generated using the specification information input from the user for an implant structure not registered in the library, and by registering it in the library, the 3D model of the custom implant structure may be utilized in implant simulation.

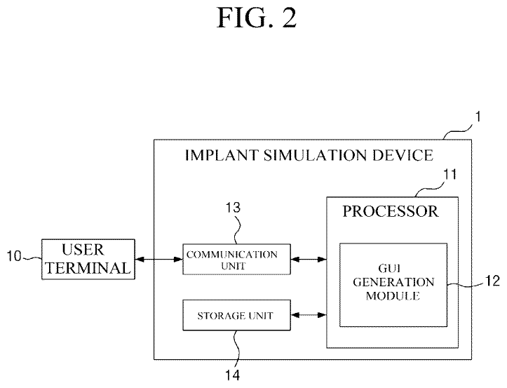

FIG. 2 is a block diagram illustrating the configuration of an implant simulation device according to an embodiment of the present disclosure.

An implant simulation device 1 according to an embodiment of the present disclosure may include a communication unit 13, a storage unit 14, and a processor 11, and may be connected to a user terminal 10 and an external device (not shown) through the communication unit 13.

Here, the user terminal 10 is a terminal device of a user or practitioner who wishes to perform implant simulation using software installed on the device, and may be implemented as, for example, a personal computer (PC), laptop computer, tablet computer, smartphone, etc. The external device (not shown) is a device that stores and manages CT images and panoramic images of the patient's oral cavity, and may be implemented as a computing device such as a server or PC.

The implant simulation device 1 is a device that provides an interface for registering a 3D model in the library and performing a series of operations for implant simulation in response to the request from the user terminal 10, and may be implemented as, for example, a computing device such as a server or PC.

The communication unit 13 communicates with the user terminal 10 and the external device using wired or wireless communication methods. The communication unit 13 may communicate with the user terminal 10 and the external device using wired communication methods such as Ethernet or wireless communication methods such as Wi-Fi or Bluetooth. The communication method of the communication unit 13 is not limited to these and other communication methods may be used for communications.

The storage unit 14 may store data related to implant simulation performed in response to the request from the user terminal 10 and store specification information and 3D models of each of multiple implant structure provided by various manufacturers in the library.

In addition, for a custom implant structure for which a 3D model is not provided by the manufacturer, the storage unit 14 may generate a 3D model based on the specification information regarding the custom implant structure input through the user terminal 10, and save the specification information and 3D model of the custom implant structure in the implant library.

The processor 11 may include a GUI generation module 12 and may further include additional modules for generating a 3D model of an implant structure.

The processor 11 receives a request for implant simulation from the user terminal 10 and performs simulation operations in response to the request from the user terminal 10.

The processor 11 provides a GUI for inputting specification information regarding the implant structure to the user terminal 10 and receives specification information regarding the implant structure through user input on the GUI. In this case, the implant structure may be a new implant product for which a 3D model is not provided by the manufacturer, or an implant product for which the library has not been updated as the product has been upgraded, and may include at least one of an implant or an abutment.

The processor 11 uses the received specification information regarding the implant structure to generate a 3D model of the implant structure and saves the generated specification information and 3D model of the implant structure in the implant library of the storage unit 14, for example, in stereolithography (STL) format.

The GUI generation module 12 provides a GUI for entering specification information regarding the implant structure.

When the implant structure is an implant, the GUI generation module 12 may provide an input screen for entering specification information such as the coronal diameter, a length, and the apical diameter of the implant and height of a hex.

Additionally, when the implant structure is an abutment, the GUI generation module 12 may provide an input screen for entering specification information such as the reference diameter of the abutment, a length from the reference diameter to the most coronal end, a length from the reference diameter to the most apical end, an axial inclination angle, and a gingival angle.

As described above, according to an embodiment of the present disclosure, the implant simulation device 1 may generate 3D models of custom implants and abutments using only the specification information regarding the implants and abutments for which 3D models are not provided, and perform implant simulation using them in software providing implant simulation functionality.

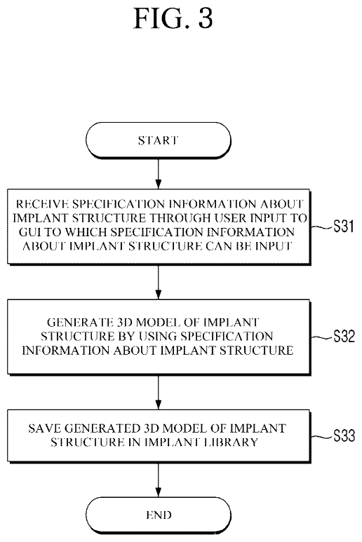

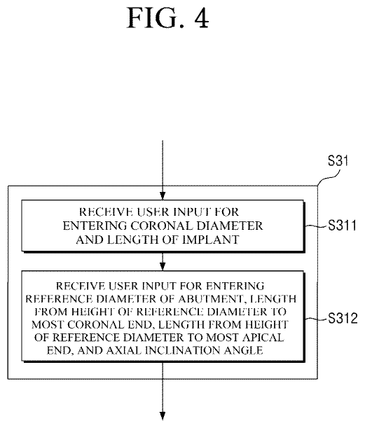

FIGS. 3 and 4 are flowcharts illustrating an implant simulation method according to another embodiment of the present disclosure.

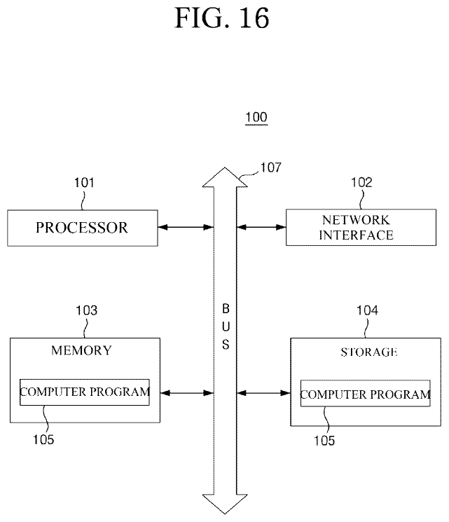

An implant simulation method according to an embodiment of the present disclosure may be executed by a computing device 100 shown in FIG. 16, and may be executed, for example, by an implant simulation device 1. The computing device 100 executing the method according to the present embodiment may be a computing device equipped with an application execution environment. Description of the subject to perform some operations included in the method according to the present disclosure may be omitted, and in such cases, it should be noted that the computing device 100 is the subject.

Referring to FIG. 3, first, in operation S31, the implant simulation device 1 provides a GUI for entering specification information regarding an implant structure and receives the specification information regarding the implant structure through user input on the GUI. The implant structure may be, for example, an implant or an abutment.

In one embodiment, referring to FIG. 4, operation S31 may include operation S311 of receiving a user input for entering the coronal diameter and the length of the implant, and operation S312 of receiving a user input for entering the reference diameter of the abutment, the length from the reference diameter to the most coronal end, the length from the reference diameter to the most apical end, and the axial inclination angle. In this case, both operation S311 and operation S312 may be performed, or only one of the operations may be performed.

In one embodiment, operation S311 may further include an operation of receiving a user input for entering the apical diameter of the implant to create the implant in a tapered shape.

In one embodiment, operation S311 may further include an operation of receiving a user input for entering a height of a hexagonal-shaped hex protruding at the top when the implant is of an external joint (or butt joint) type where the abutment is inserted and coupled externally toward the top of the implant.

In one embodiment, operation S312 may include an operation of receiving a user input for entering a gingival angle for coupling to a lower part of the abutment, rather than receiving an input for a length from the height of the reference diameter to the most apical end. In this case, the gingival angle may be determined as an angle of a cylindrical cone connecting the outermost part of the uppermost circle of the implant and the outermost part of the diameter of the abutment.

Operation S312 may further include displaying notification information when the position of a diameter area of the abutment changes according to the input gingival angle. In this case, depending on the gingival angle, the position of the diameter area of the abutment may change to one of the following states: an equigingival margin located at the same level as the top of gingiva, a supragingival margin located above the top of the gingival, and a subgingival margin located below the top of the gingiva.

In one embodiment, operation S312 may further include an operation of receiving a user input for entering an abutment angle and an angle of inclination of an abutment to create the abutment in a shape that is asymmetrical in the left and right sides.

Next, in operation S32, the implant simulation device 1 generates a 3D model of the implant structure using the received specification information regarding the implant structure.

In operation S33, the implant simulation device 1 saves the generated 3D model of the implant structure in the implant library. In this case, operation S33 may include an operation of registering the specification information and the 3D model of the implant structure in the pre-stored implant library, and an operation of displaying the registered specification information and 3D model of the implant structure on the list in the implant library.

Finally, in operation S34, the implant simulation device 1 performs implant simulation using the specification information and 3D model of the implant structure registered in operation S33.

As described above, according to an embodiment of the present disclosure, a convenient user interface may be provided to register 3D models of various types of implants and abutments not yet registered in the library in the software providing implant simulation functionality.

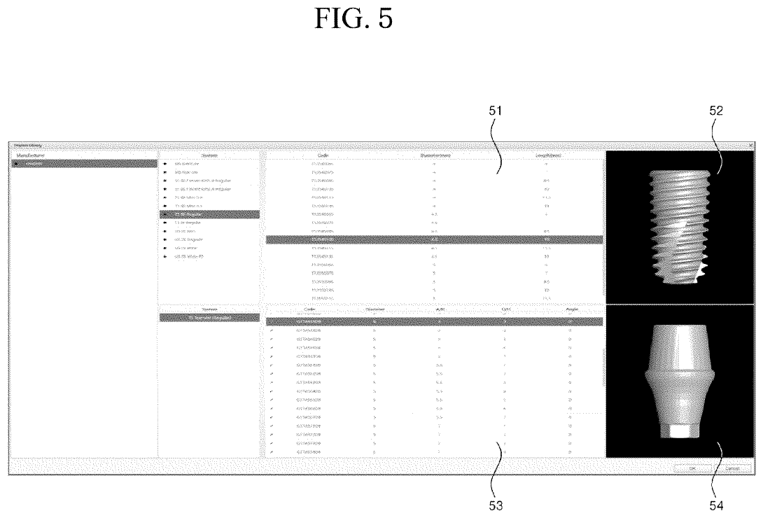

FIG. 5 illustrates an example of an implant library display screen according to some embodiments of the present disclosure. Referring to FIG. 5, in the software providing implant simulation functionality, surgical planning may be performed by selecting an implant or an abutment registered in the implant library. The implant library stores specification information and 3D models of implant products from various manufacturers.

In the illustrated example, the display screen of the implant library may display a list of implant libraries, including specification information 51 regarding each of multiple implants and a rendered image 52 of a 3D model corresponding to each implant. Similarly, the display screen of the implant library may include a list of abutment libraries, including specification information 53 regarding each of multiple abutments and a rendered image 54 of a 3D model corresponding to each abutment.

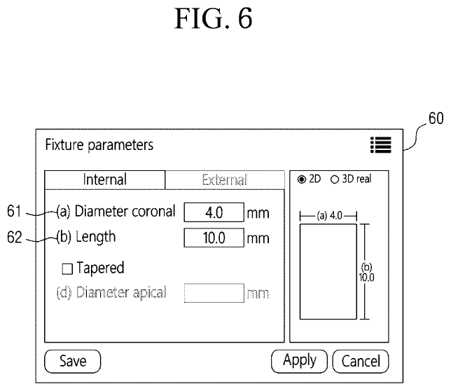

FIG. 6 illustrates an example of a screen for entering information to create a custom implant of an internal joint type according to some embodiments of the present disclosure. Referring to FIG. 6, the implant simulation device 1 provides an input screen 60 for entering specification information to register a custom implant of an internal joint type in the library.

In the illustrated example, the input screen 60 provides an interface for entering the coronal diameter 61 and the length 62 of the implant in the case of the internal joint type where the abutment is inserted and coupled internally to the implant. Here, the length 62 of the implant is based on the length of the line connecting the center points of the top and bottom of the implant.

The implant simulation device 1 may automatically generate a 3D model of the custom implant using the specification information entered through the input screen 60. Additionally, the implant simulation device 1 may register the specification information and 3D model of the implant in a custom implant library.

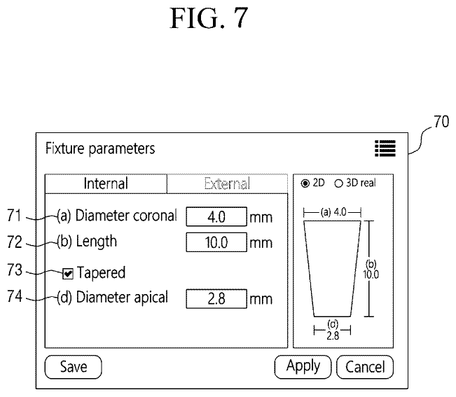

FIG. 7 illustrates an example of a screen for entering information to register a custom implant in a tapered shape according to some embodiments of the present disclosure. Referring to FIG. 7, the implant simulation device 1 provides an input screen 70 for entering specification information to register a custom implant in a tapered shape in the library.

In the illustrated example, the input screen 70 provides an interface for entering whether the implant is tapered 73 and the apical diameter 74 of the implant, in addition to the coronal diameter 71 and the length 72 of the implant, in the case where the implant tapers towards the bottom. However, the apical diameter 74 of the implant cannot be larger than the coronal diameter 71, and if a larger value is entered, a warning message may be displayed to alert the user or a message may be provided to guide the user to enter a value smaller than a certain value.

Accordingly, the implant simulation device 1 may generate a 3D model of the custom implant in a tapered shape using the specification information entered through the input screen 70 and register it in the implant library.

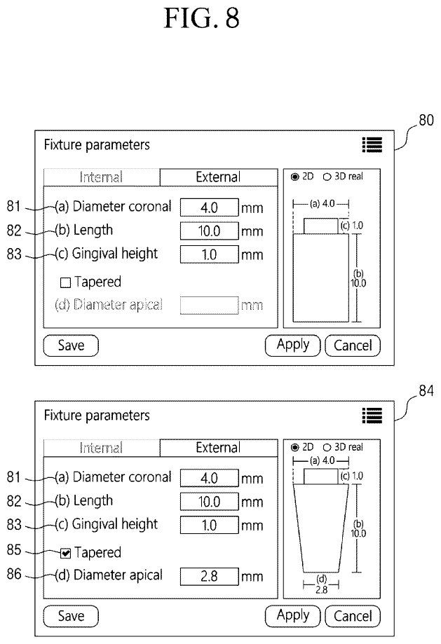

FIG. 8 illustrates an example of a screen for entering information to create a custom implant of an external joint type according to some embodiments of the present disclosure. Referring to FIG. 8, the implant simulation device 1 provides input screen 1 80 or input screen 2 84 for entering specification information to create a 3D model of a custom implant of an external joint type.

In the illustrated example, input screen 1 80 provides an interface for entering the height of a hex 83, in addition to the coronal diameter 81 and the length 82 of the implant, in the case of the external joint (or butt joint) type where the abutment is inserted and coupled externally to the implant. Here, the hex refers to a structure corresponding to the engagement part between the abutment and the implant, protruding externally from the top of the implant, not toward the inside of the implant.

On the other hand, in the case where the implant is of an external joint type and tapers towards the bottom, input screen 2 84 provides an interface for entering whether the implant is tapered 85 and the apical diameter 86 of the implant, in addition to the coronal diameter 81 and the length 82 of the implant and the height 83 of a hex.

Accordingly, the implant simulation device 1 may generate the 3D model of the custom implant of the external joint type using the specification information entered through input screens 80 and 84 and register it in the implant library.

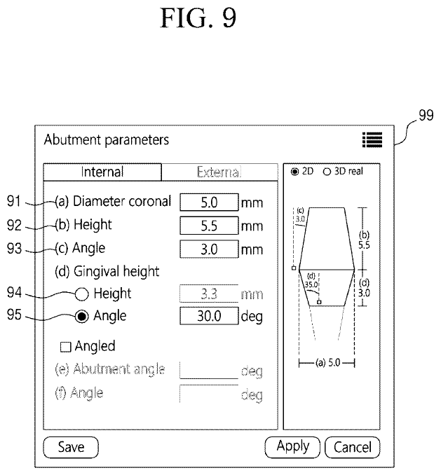

FIG. 9 illustrates an example of a screen for entering information to create a custom abutment according to some embodiments of the present disclosure.

Referring to FIG. 9, the implant simulation device 1 provides an input screen 99 for entering specification information to create a 3D model of a custom abutment of the internal joint type.

In the illustrated example, in the case of the internal joint type where an abutment is inserted and coupled internally to an implant, the input screen 99 provides an interface for entering a reference diameter 91 of an abutment, the length from the height of the reference diameter 92 to the most coronal end, the axial inclination angle 93, and the length 94 from the height of the reference diameter to the most apical end.

Furthermore, the apical diameter of the abutment may be automatically determined to be the diameter of the portion in contact with the implant without requiring separate user input. Similarly, the coronal diameter of the abutment may also be automatically determined based on the height 92 from the reference diameter to the most coronal end, which is input to the input screen 99, and the axial inclination angle 93, without requiring separate user input.

In one embodiment, an interface may be provided for entering a gingival angle 95 for coupling to a lower side of the abutment, rather than entering the length 94 from the reference diameter to the most apical end to the input screen 99.

In one embodiment, the gingival angle 95 input through the input screen 99 may be limited to 30 degrees or more. For example, if a value less than 30 is input to an input field for the gingival angle 95 on the input screen 99, a warning message may be displayed to prompt the user to enter a value of 30 or more.

According to the embodiments as described above, the implant simulation device 1 may automatically generate a 3D model of a custom abutment using the specification information regarding the abutment input through the input screen 99. Additionally, the specification information and 3D model of the abutment may be registered in the abutment library.

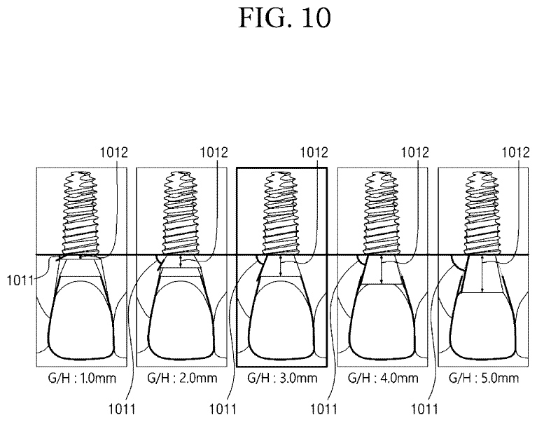

FIG. 10 illustrates an example where the gingival angle changes when the length from the diameter of an abutment to the most coronal end of an implant changes according to the gingival angle, or when the length from a set diameter of the abutment to the most coronal end of the implant changes. Referring to FIG. 10, when an input value of a gingival angle 1011 changes, a length 1012 from the diameter to the most apical end is automatically changed accordingly. In this case, to maintain the input gingival angle 1011, the position of the diameter area, i.e., margin area, of the abutment may change.

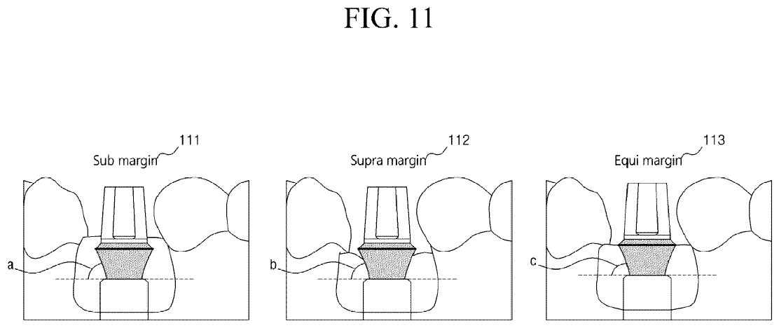

Specifically, in the example illustrated in FIG. 11, depending on the input gingival angle 1011, the margin area of the abutment may change to one of the following states: an equigingival margin 113 located at the same level as the top of the gingiva, a supragingival margin 112 located above the top of the gingiva, and a subgingival margin 111 located below the top of the gingiva.

In this case, if the position of the abutment's margin area changes according to the input gingival angle 1011, a guidance message may be displayed on the screen to allow the user to confirm the change.

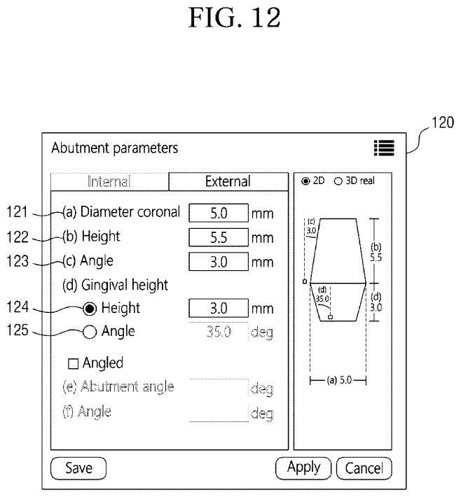

FIG. 12 illustrates an example of a screen for entering information to create a custom abutment of an external joint type according to some embodiments of the present disclosure. Referring to FIG. 12, the implant simulation device 1 provides an input screen 120 for entering specification information to generate a 3D model of a custom abutment of the external joint type.

In the illustrated example, in the case of the external joint type where an abutment is inserted and coupled externally to an implant, the input screen 120 provides an interface for entering a reference diameter 121 of the abutment, a length 122 from the height of the reference diameter to the most coronal end, an axial inclination angle 123, and a length 124 from the height of the reference diameter to the most apical end of the abutment In this case, instead of the length from the height 124 of the reference diameter to the most apical end, a gingival angle 125 may be input.

As described above, the implant simulation device 1 provides an interface to generate a 3D model of a custom abutment for the external joint type shown in FIG. 12 or the internal joint type shown in FIG. 9. In this case, GUI elements such as tabs or buttons may be provided to allow selection between external joint type and internal joint type.

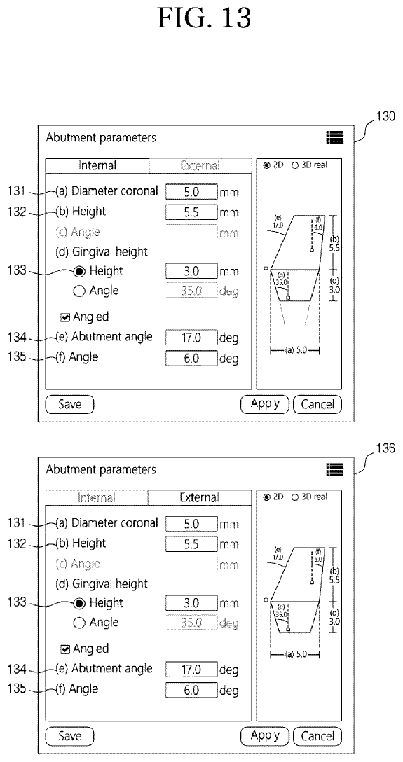

FIG. 13 illustrates an example of a screen for entering information to create a custom abutment in a shape that is asymmetrical in the left and right sides according to some embodiments of the present disclosure. Referring to FIG. 13, the implant simulation device 1 provides input screen 1 130 for entering specification information to generate a 3D model of a custom abutment in a shape asymmetrical in the left and right sides in the internal joint type, or input screen 2 136 for generating an abutment library in a shape asymmetrical in the left and right sides in the external joint type. The custom abutment in a shape asymmetrical in the left and right sides may be applied when a significant compensation angle using an abutment is required based on the placement result of implants and crowns.

In the illustrated example, input screen 1 130 corresponds to a case where the abutment is of the internal joint type, where the abutment is inserted and coupled internally to the implant, and has a shape asymmetrical in the left and right sides, while input screen 2 136 corresponds to a case where the abutment is of the external joint type, where the abutment is inserted and coupled externally to the implant, and has a shape asymmetrical in the left and right sides. In the illustrated example, input screen 1 130 and input screen 2 136 each provide an interface for entering a reference diameter 131 of the abutment, a length 132 from the height of the reference diameter to the most coronal end, a length 133 from the height of the reference diameter to the most apical end, and additionally, an abutment angle 134 and an angle 135 of inclination of abutment. In this case, the abutment angle 134 represents the compensation angle for the entire abutment, and the angle 135 of inclination of abutment corresponds to the compensation angle on the opposite side of the abutment angle 134. The abutment angle 134 may be directly input by the user, or may be calculated by software based on angle information of the reference axes of the implant and crown, and then provided to the user.

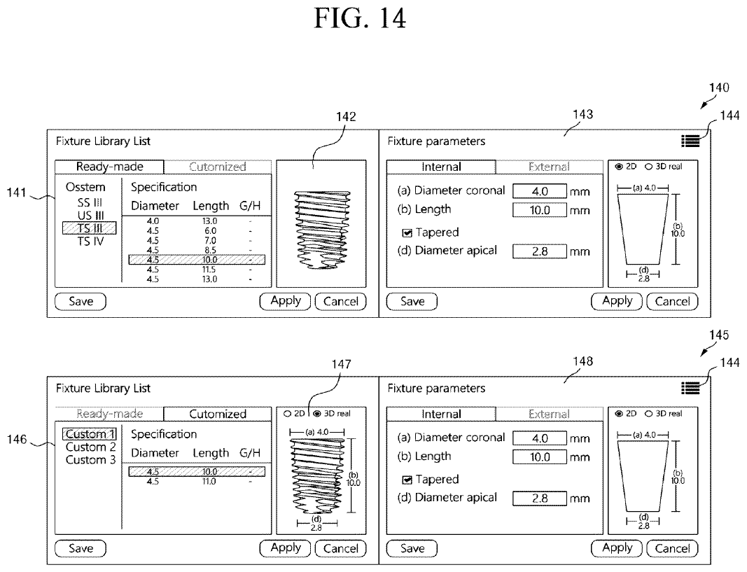

FIG. 14 illustrates an example of a screen for registering a 3D model of a virtual implant in a library according to some embodiments of the present disclosure. Referring to FIG. 14, upon receiving input of specification information regarding an implant or abutment through a GUI, the implant simulation device 1 may automatically generate a 3D model using the input specification information, and register the generated 3D model and specification information in the implant library. In this process, the 3D model generated using the specification information regarding the implant or abutment may be added to an existing library of ready-made implants and saved, or it may be saved in a separately created library.

In the illustrated example, when an activation button 144 is selected on a library screen 140 of ready-made implants that has already been constructed, the specification information regarding various types of ready-made implant models is displayed in list form 141 on the left side of the screen 140, and the exemplary shape of each model is displayed in a rendered image 142. At this time, on the right side of the screen 140, detailed specifications of the selected model and an input screen 143 for modifying them are provided.

Meanwhile, when the activation button 144 is selected on the library screen 145 to add an unregistered implant to the library, specifications of various types of custom implants are displayed in list form 146 for each model on the left side of the screen 145, and the exemplary shape of each model is displayed in the rendered image 147. At this time, on the right side of the screen 145, detailed specifications of each custom model registered by the user and an input screen 148 for modifying them are provided.

In this manner, the implant simulation device 1 may distinguish and display the library list of ready-made implants stored in the software and the custom implants registered by user input.

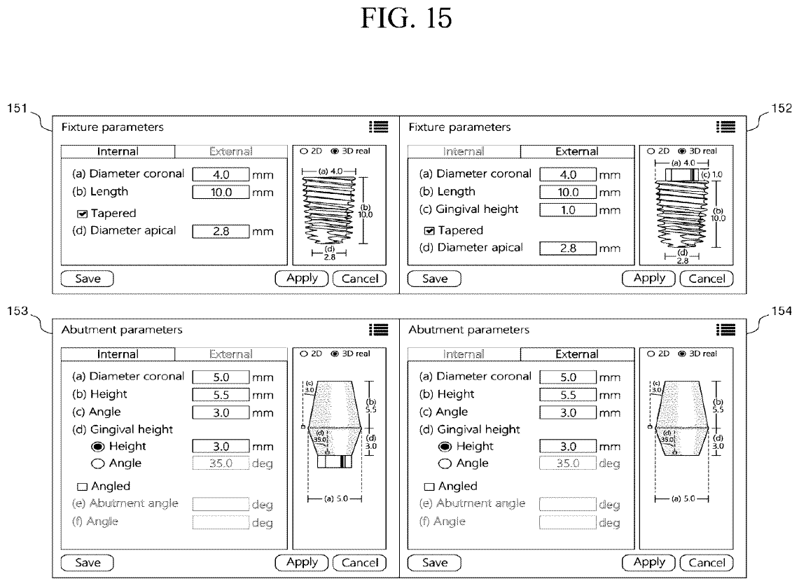

FIG. 15 illustrates an example of an interface screen for generating a 3D model of an implant structure according to some embodiments of the present disclosure. Referring to FIG. 15, the implant simulation device 1 may render and display a custom implant in 3D or 2D format using the specification information regarding the implant of the internal joint type input through input screen 1 151. Additionally, the implant simulation device 1 may render and display a custom implant in 3D or 2D format using the specification information regarding the implant of the external joint type input through input screen 2 152.

Furthermore, the implant simulation device 1 may render and display a custom abutment in 3D or 2D format using the specification information regarding the abutment of the internal joint type or external joint type input through input screen 3 153 or input screen 4 154.

The custom implant and custom abutment generated as described above in 3D format may be checked through rotation and may be saved as 3D model data in STL format. FIG. 16 is a diagram illustrating a hardware configuration of an exemplary computing device by which methods according to some embodiments of the present disclosure can be implemented. As illustrated in FIG. 16, the computing device 100 may include at least one processor 101, a bus 107, a network interface 102, a memory 103 configured to load a computer program 105 executed by the processor 101, and a storage 104 configured to store the computer program 105. However, only the components relating to the embodiment of the present disclosure are illustrated in FIG. 16. Therefore, those of ordinary skill in the art to which the present disclosure pertains should understand that the computing device 100 may further include general-purpose components other than the components illustrated in FIG. 16.

The processor 101 controls the overall operation of each configuration of the computing device 100. The processor 101 may be configured to include at least one of a central processing unit (CPU), a micro processor unit (MPU), a micro controller unit (MCU), a graphic processing unit (GPU), or any other processor widely known in the art to which the present disclosure pertains. Also, the processor 101 may perform computations for at least one application or program for executing methods/operations according to various embodiments of the present disclosure. The computing device 100 may include one or more processors.

The memory 103 stores various types of data, commands, and/or information. The memory 103 may load one or more programs 105 from the storage 104 in order to execute the methods/operations according to various embodiments of the present disclosure. For example, when the computer program 105 is loaded into the memory 103, logic (or modules) may be implemented on the memory 103. An example of the memory 103 may be a random access memory (RAM), but the memory 103 is not limited thereto.

The bus 107 provides communication functionality between the components of the computing device 100. The bus 107 may be implemented as various types of buses, such as an address bus, a data bus, and a control bus.

The network interface 102 supports wired/wireless Internet communication of the computing device 100. The network interface 102 may also support various communication methods other than the Internet communication. To this end, the network interface 102 may be configured to include a communication module widely known in the art to which the present disclosure pertains.

The storage 104 may non-temporarily store one or more computer programs 105. The storage 104 may be configured to include a nonvolatile memory such as a flash memory, a hard disk, a removable disk, or any other computer-readable recording media widely known in the art to which the present disclosure pertains.

The computer programs 105 may include one or more instructions implementing the methods/operations according to various embodiments of the present disclosure. When the computer program 105 is loaded on the memory 103, the processor 101 may execute the one or more instructions to perform the methods/operations according to various embodiments of the present disclosure.

So far, various embodiments and effects thereof, in which the technical features of the present disclosure have been implemented, have been described with reference to FIGS. 1 to 16. Effects according to the technical features of the present disclosure are not limited to the above-described effects, and other effects not described will be clearly understood by those skilled in the art from the following description.

The technical features of the present disclosure described above may be implemented as computer readable codes on a computer-readable medium. Examples of the computer-readable recording media may include removable recording media (a compact disc (CD), a digital versatile disc (DVD), a Blu-ray Disc, a universal serial bus (USB) storage device, or a removable hard disk) or non-removable recording media (a read-only memory (ROM), a random access memory (RAM), or a built-in hard disk). The computer program recorded on the computer-readable recording medium may be transmitted to another computing device through a network such as the Internet and installed in the other computing device, thereby being used in the other computing device.

In the above description, it is described that all the components constituting the embodiments of the present disclosure are combined or operated as one, but the technical features of the present disclosure are not limited to these embodiments. That is, within the scope of the present disclosure, all of the components may be selectively combined and operated in one or more combinations.

Although the operations are shown in a specific order in the drawings, it should not be understood that the operations must be performed in the specific order or sequential order shown, or that all the illustrated operations must be executed to achieve the desired results. In certain circumstances, multitasking and parallel processing may be advantageous. Moreover, the separation of the various configurations in the embodiments described above should not be understood as requiring that separation, and it should be understood that the described program components and systems may generally be integrated together into a single software product or packaged into multiple software products.

In concluding the detailed description, those skilled in the art will appreciate that many variations and modifications can be made to the exemplary embodiments without substantially departing from the principles of the present disclosure. Therefore, the disclosed exemplary embodiments of the disclosure are used in a generic and descriptive sense only and not for purposes of limitation. The scope of the present disclosure should be interpreted by the claims below, and any technical features within the scope equivalent to the claims should be interpreted as falling within the scope of the technical features defined by the present disclosure.

1. A method performed by a computing device, the method comprising the steps of:
receiving specification information regarding an implant structure through a user input on a graphical user interface (GUI) to which the specification information regarding the implant structure can be input;
generating a three-dimensional (3D) model of the implant structure by using the received specification information regarding the implant structure; and
saving the generated 3D model of the implant structure in an implant library.

2. The method of claim 1, wherein:
the implant structure comprises an implant; and
the receiving of the specification information regarding the implant structure comprises receiving a user input for entering a coronal diameter and a length of the implant.

3. The method of claim 2, wherein the receiving of the specification information regarding the implant structure further comprises receiving a user input for entering an apical diameter of the implant to create the implant in a tapered shape.

4. The method of claim 2, wherein the receiving of the specification information regarding the implant structure further comprises receiving a user input for entering a height of a hexagonal-shaped hex protruding at a top when the implant is of an external joint or butt joint type.

5. The method of claim 1, wherein:
the implant structure comprises an abutment; and
the receiving of the specification information regarding the implant structure comprises receiving a user input for entering a reference diameter of the abutment, a length from a height of the reference diameter to the most coronal end, a length from the height of the reference diameter to the most apical end, and an axial inclination angle.

6. The method of claim 5, wherein the receiving of the specification information regarding the implant structure comprises receiving a user input for entering a gingival angle for coupling to a lower part of the abutment, rather than receiving an input for a length from the height of the reference diameter to the most apical end.

7. The method of claim 6, wherein the receiving of the user input for entering the gingival angle for coupling to the lower side of the abutment comprises receiving an input for an angle of a cylindrical cone connecting an outermost part of an uppermost circle of the implant and an outermost part of the diameter of the abutment.

8. The method of claim 6, further comprising the step of displaying notification information when a position of a diameter area of the abutment changes according to the input gingival angle.

9. The method of claim 6, further comprising the step of changing a state of a diameter area of the abutment to any one of an equigingival margin located at the same level as a top of gingiva, a supragingival margin located above the top of the gingiva, and a subgingival margin located below the top of the gingiva, according to the input gingival angle.

10. The method of claim 5, wherein the receiving of the specification information regarding the implant structure further comprises receiving a user input for entering an abutment angle and an angle of inclination of an abutment to create the abutment in a shape that is asymmetrical in the left and right sides.

11. The method of claim 1, wherein the saving of the generated 3D model of the implant structure in the implant library comprises:
registering the specification information regarding the implant structure and the 3D model in a pre-stored implant library; and
displaying the registered specification information regarding the implant structure and the registered 3D model on a list of the implant library.

12. An implant simulation device comprising:
at least one processor;
a communication interface configured to communicate with an external device;
a memory configured to load a computer program executed by the processor; and
a storage configured to store the computer program,
wherein the computer program comprises instructions to perform an operation of receiving specification information regarding an implant structure through a user input on a graphical user interface (GUI) to which the specification information regarding the implant structure can be input, an operation of generating a three-dimensional (3D) model of the implant structure by using the received specification information regarding the implant structure, and an operation of saving the generated 3D model of the implant structure in an implant library.
